SEQUENCE LISTING (3 sequences)

SEQ ID NO: 1 - protein, 1141 aa, Streptococcus suis
MNIQERFSLRKSAVGLVSVSLLCAIYTSTVAADTVVTGVNEIIEESQVKDEVSIESEKNESLDGSNIEIVEEIADNIPSPVIAEGEVAVEMKVDRGTENVVSRNDTEVTTSEQNQIEVTETKEILNQTSYQTESGEQRQIIWAHGITPPAMEQSGGFVKEKYGDYLNYTAPFEAGKGYYDTNKSLNASFIDLNLCFAAVSSNMVHWWLEQNSSYVERYLKEKKGTVNVEENYAITDLRRYINSFQNQQNSRVFDMFKTYYGYRTNGFVSDALVDLFINGYKPKAQGGVNLEDSQLVPDSRGGFFYDVFKEKKLTNRIFSGSYERFGEDVRTVLESKGLLGLTYRTLGYATHIVTVWGAEYDNQGKIKAVYITDSDDQQEQIGLKRMGITRDASGNPRLNNHMKNNSAGALLDYVHTIRLGQDLWEEYFNPLAKAKETASQTLADTKKALDLSIQGQSELPESMRLIYLEKLNNLYNQGILSIQKAESSEMLSGALENGLNSLKSLDFPISEVGNALAPDLPVGDRSTVSDVDSLSSQETSSTNLEADTENAGIIADGTNQLHFPVEAQTTSSVEAEGDNVFEQEADTLPIIIENKDEFGSELSRNMQTSETDSLVVAVEEDVKNDEVAQVEELLESEKVENQSSELLSDTLIVESANDKEEDRVEAVVSEQPDSIPHQNVEISLVEPTNVETETVVTPINDAATPHGSPTYIDNSVTESVATPLEKDSIQAGETEIAEPTSSESTNVETETVVTPVNDVATPHGSPTYIDNSVTESVATPLEKDSIQAGETEIAEPTSSESTNVETETVVTPVNDVATPHGSPTYIDNSVTESVATPLEKDSIQAGETEIAEPTSSESTSVEAELVDNSEIHAATSSVTPCGSSAYADGSTTESVATPLEKDSIQTGNTEIAEPTSSKSTNVEAASVDNSEIHADASLTAVSSVNLDNPVIEPVAISLIGSKRDTNAEVEVSSLSKREVRKTNTDGLISVQSKVIKKELLESSLAEAGSPLLEATIAQSSNSNSTEIGMSYQNTVLLESNNTERQVSKAEIVMEHKETELVETVSSASEPVVLVENISQTSNNTIESGKNMGVQSQAGAKQILGVEQSSKVSTPTSRQIMGVGLLTLVLGSALGLLKKRRK

SEQ ID NO: 2 - protein, 980 aa, Artificial Sequence
  Modified IgM protease of Streptococcus suis serotype 7, without signal sequence and 25 aa tail, but with His-tag
MDTVVTGVNEIIEESQVKDEVSIESEKNESLDGSNIEIVEEIADNIPSPVIAEGEVAVEMKVDRGTENVVSRNDTEVTTSEQNQIEVTETKEILNQTSYQTESGEQRQIIWAHGITPPAMEQSGGFVKEKYGDYLNYTAPFKAGKGYYDTNKSLNASFIDLNLCFAAVSSNMVHWWLEQNSSYVERYLKEKKGTVNVGENYAITDLRRYIDSFQDQQNSRVFDMFKTYYGYRTNGFVSDALVDLFINGYKPKVQGGVNLEDSQLVPDSRGGFFYDVFKEKKLTNRIFSGSYERFGEDVRTVLESKGLLGLTYRTLGYATHIVTVWGAEYDNQGKIRAVYITDSDDQQEQIGLKRMGITRDASGNPRLNNHVKNNSAGALLDYVHTIRLGQDLWEEYFNPFAKAKEIASQILADRKKALVLSIQGQSELPESMRLIYLEKLNNLYNQGILSIQKTESSEMLSGALENGLNSLKSLDFPISEVGNALAPDLSVGDRSTVSDVDSLSSQETSSTNLEADTENAGIIADGTNQLHFPVEAQTTSSVEAEGDNVFEQEADTLPIIIENKDEFGSELSGNMQTSETDSLVVAVEEDVKNDEVDQVEKLLESEKVENQSSELLSDTLIVEGANDKEEDRVEAVVSEQPDSIPHQNVEISPVEPMNVETESVVTPINDAATPHGFPMYIDNSVTESVATPLEKDSIQAGETEIAEPTSSESTSVEAELVDNSEIHSATSSVTPRGSSAYADSSTTESVATLLEKDSIQAGETEIAEPTSSKSTNVEAASVDNSEIHADTSLTAVSSVNLDNPVIEPVAIPLIGSKRDTNAEVEVSSLSKREVRKPNTEGLISVQSKVIKKELLESSLVEAGSPLLEATIAQSSNSNSTEIGMSYQNTVLLESNNTERQVSKAEIVIEHKETELVETVSSASEPVVLVENISQTSNNTIESGKNMGVQSQAGAKQILGIEQSSKVSTPTHHHHHHHHHH

SEQ ID NO: 3 - protein, 980 aa, Artificial Sequence
  Modified IgM protease of Streptococcus suis serotype 1, without signal sequence and 25 aa tail, but with His-tag
MDTVTTRENLTIEELQEKEEASTESEKLGSLDDSSSEIVEKVVDNIPSSIGAERDGAVEMKVDRGTENVVSRNDTEVTTSEQNQIEVTETKEILNQTSYQTESGEQRQIVWAYGITPPVMEQKGGFVKEKYGDYLNYTAPFEAGKGYYDTNKSLNASFIDLNLCFAAVSSNMVHWWLEQNSSYVERYLKEKKGIVNVEENYAITDIRRYINSFQNQQNSRVFDMFKTYYGYRTNGFVSDALVDLFINGYKPKAQGGVNLEDSHLVPDSRGGFFYDVFKEKKLTNRIFSGSYERFGEDVRTVLESKGLLGLTYRTLGYATHIVTVWGAEYDNQGKIRAVYITDSDDQQEQIGLKRMGITRDASGNPRLNNHVKNNSAGALLDYVHTIRLGQDLWEEYFNPLAKSKEIASQTLADTKKALDLSIQGQSELPESMRLIYLEKLNNLYNQGILSIQKAESSEMLSGALENGLNSLKSLDFPISEVGNALAPDLPVGDRSTVSDVDSLSSQETSSTNLEVDTENAGLIADGADQLHFPVEVQTTSFVEAEGDNVFEQEADTLPIIIENKDEFGSELSRNMQTSETDSLVVAVEEDVKNDEVAQVEELLELEKVENQSSELLSDTLIVEGANDKEEDRVEAVVSEQPDSIPHQNVEISLVEPTNVETESVVTPINDVATPHGSPTYIDNSVTESVATPLEKDSIQAGETENAEPTSSESTSVEAELVDNSEIHAATSSVTPCGSSAYADSSATESVATPLEKDSIQTGNTEIAEPTSSKSTNVEAASVDNSEIHADASLTAVSSVNLDNPVIEPVAISLIGSKRDTNAEVEVSSLSKREVRKPNTDGLISVQSKVIKKELLESSLVEAGSPLLEATIAQSSNSNSTEIGMSYQNTVLLESNNTERQVSKAEIVMEHKETELVETVSSASEPVVLVENISQTSNNTIESGKNMGVQSQAGAKQILGVEQSSKVSTPTHHHHHHHHHH

(19) United States
(12) Patent Application Publication
(10) Pub. No.: US 2024/0335520 A1
(43) Pub. Date: Oct. 10, 2024

(54) A VACCINE FOR PROTECTION AGAINST STREPTOCOCCUS SUIS OF VARIOUS SEROTYPES

(71) Applicants: Intervet Inc., Rahway, NJ (US); INTERVET INTERNATIONAL B.V, AN Boxmeer (NL)

(72) Inventor: Antonius Arnoldus Christiaan Jacobs, Kessel (NL)

(73) Assignees: Intervet Inc., Rahway, NJ (US); INTERVET INTERNATIONAL B.V, AN Boxmeer (NL)

(21) Appl. No.: 18/293,378

(22) PCT Filed: Jun. 29, 2022
(86) PCT No.: PCT/EP2022/067890

§ 371 Date: Jan. 30, 2024

(30) Foreign Application Priority Data
Aug. 3, 2021 (EP) 21189277.3

(51) Int. Cl. A61K 39/09; A61K 38/48; A61K 39/00; A61P 31/04

(52) U.S. Cl. CPC A61K 39/092; A61K 38/4873; A61P 31/04; A61K 2039/52; A61K 2039/552

(57) ABSTRACT

The present invention pertains to a vaccine comprising in combination an IgM protease antigen of Streptococcus suis serotype (7), a Streptococcus suis bacterin serotype (9), sequence type (16), and a pharmaceutically acceptable carrier. The invention also pertains to a combination of an IgM protease antigen of Streptococcus suis serotype (7), and a Streptococcus suis bacterin serotype (9), sequence type (16), for use in a method to protect a pig against a pathogenic infection with Streptococcus suis and to a method for protecting pigs against a pathogenic infection with Streptococcus suis, by administering to the pigs an IgM protease antigen of Streptococcus suis serotype (7) and a Streptococcus suis bacterin serotype (9), sequence type (16).

### GENERAL FIELD OF THE INVENTION

The invention pertains to the protection of pigs against a pathogenic infection with Streptococcus suis bacteria of various serotypes, in particular the most prevalent serotypes 1, 2, 7 and 9.

### BACKGROUND OF THE INVENTION

Streptococcus suis (S. suis) is one of the principal etiologic agents of contagious bacterial disease in pigs. The pathogen can cause a variety of clinical syndromes including meningitis, arthritis, pericarditis, polyserositis, septicaemia, pneumonia and sudden death. S. suis is a gram-positive facultatively anaerobic coccus, originally defined as Lancefield groups R, S, R/S or T. Later, a new typing system based on the type-specific capsular polysaccharide antigens located in the cell wall was proposed. This led to a system comprising 35 serotypes (Rasmussen and Andresen, 1998, “16S rDNA sequence variations of some Streptococcus suis serotypes”, Int. J. Syst. Bacteriol. 48, 1063-1065) of which serotypes 1, 2, 7 and 9 are currently the most prevalent, especially in Europe. However, it is recognised that the capsular serotype is a poor marker of virulence. Therefor an alternative system to helping understand the epidemiology of S. suis infection and the biological relevance of the serotyping approach was developed, i.e. the so called multilocus sequence typing (MLST), as described by King et al. in the Journal of Clinical Microbiology, October 2002, p. 3671-3680 (Development of a Multilocus Sequence Typing Scheme for the pig pathogen Streptococcus suis: Identification of virulent clones and potential capsular serotype exchange”). In that study 92 sequence types were identified, of which ST complexes ST1, ST27 and ST87, each containing multiple sequence types, dominate the population. See also the Streptococcus suis MLST website (https://pubmlst.org/ssuis/) sited at the University of Oxford (Jolley et al. Wellcome Open Res 2018, 3:124 (site funded by the Wellcome Trust), which refers to the King et al. paper and allows for easy identification of the sequence type for any Streptococcus suis strain.

Control of Streptococcus suis in pig herd appears to be difficult. Streptococcus suis is a commensal and opportunistic pathogen of swine. Apparently, the immune system is not triggered in each and every occasion of an infection. Next to this, Streptococcus suis is a well-encapsulated pathogen and uses an arsenal of virulence factors to evade the host immune system. Together, these characteristics have challenged the development of efficacious vaccines to fight this important pathogen. An overview article has been published a few years ago, the article reviewing existing and explorative vaccines against Streptococcus suis (Mariela Segura: “Streptococcus suis vaccines: candidate antigens and progress, in Expert Review of Vaccines, Volume 14, 2015, Issue 12, pages 1587-1608). In this review, clinical field information and experimental data have been compiled and compared to give an overview of the status of vaccine development against Streptoccus suis as outlined here below.

Current commercial vaccines are mainly whole-cell bacterins. However, field reports describe difficulty in disease control and management, and specially “vaccine failures” when using bacterin vaccines are common, in particular since heterologous protection is very poor. Carrier pigs are the primary source of infection, and both vertical and horizontal transmission are involved in spread of the disease within a herd. Mixing of carrier animals with susceptible animals under stressful conditions such as weaning and transportation usually results in clinical disease. Early medicated weaning and segregated early weaning practices do not eliminate Streptococcus suis infection. Therefore, effective control measures to prevent disease will hinge on prophylactic/metaphylactic procedures (where allowed) and on vaccination. Currently, field immunization efforts have focused on the use of commercial or autogenous bacterins. These vaccine strategies have been applied to either piglets or sows. From weaning and onwards piglets are more susceptible to Streptococcus suis infections due to the stresses associated with weaning and also, the common subsequent transport. Therefore, prepartum immunization in sows is often used to try and convey passive immunity to piglets and provide protection against Streptococcus suis under these stressful circumstances early in life. Moreover, sow vaccination is less costly and labor intensive, thus representing an economical alternative to piglet vaccination. Yet, available results seem to indicate that sow vaccination with bacterins is also a matter of controversy. In many cases vaccinated sows, even when vaccinated twice before parturition, respond poorly or not at all to vaccination which results in low maternal immunity transferred to the litters. And even if maternal immunity is transferred at a sufficient level, in many cases the maternal antibodies are too low to provide protection in the most critical period of 4-7 weeks of age.

In piglets, autogenous bacterins are frequently used in the field, especially in Europe. They are prepared from the virulent strain isolated on the farm with clinical problems and applied to the same farm. One of the disadvantages of autogenous bacterins is that vaccine safety data are lacking and severe adverse reactions may occur. Sampling errors (due to using only one or two pigs or samples) may result in failure to identify a strain or serotype associated with a recent outbreak. This failure may be especially problematic in endemic herds. Finally, the most important dilemma of autogenous bacterins is that their actual efficacy has been poorly studied. As application of autogenous vaccines is empirical, it is not surprising that results obtained with these vaccines are inconsistent and often disappointing.

Other experimental vaccines are also described in the art. Kai-Jen Hsueh et al. show (“Immunization with Streptococcus suis bacterin plus recombinant Sao protein in sows conveys passive immunity to their piglets”, in: BMC Veterinary Research, BMC series—open, inclusive and trusted, 13:15, 7 Jan. 2017) that a bacterin plus subunit might be a basis for successful vaccination of sows to confer protective immunity to their piglets.

Live attenuated vaccines have also been contemplated in the art. Non encapsulated isogenic mutants of Streptococcus suis serotype 2 have been clearly shown to be avirulent. Yet, a live vaccine formulation based on a non encapsulated serotype 2 mutant induced only partial protection against mortality and failed to prevent the development of clinical signs in pigs challenged with the wildtype strain (Wisselink H J, Stockhofe-Zurwieden N, Hilgers L A, et al. “Assessment of protective efficacy of live and killed vaccines based on a non-encapsulated mutant of Streptococcus suis serotype 2.” in: Vet Microbiol. 2002, 84:155-168.)

In the last couple of years, an extensive list of antigenic or immunogenic Streptococcus suis molecules has been reported, and most of these have been discovered through immuno proteomics using either convalescent sera from infected pigs or humans and/or laboratory-produced immune sera. WO2015/181356 (IDT Biologika GmbH) has shown that IgM protease antigens (either the whole protein or the highly conserved Mac-1 domain representing only about 35% of the full protein) can elicit a protective immune response in piglets in a vaccination scheme of administering two doses of the IgM protease antigen, optionally in combination with a prime vaccination containing a bacterin. It is suggested in the '356 patent application that the IgM protease antigen, due to the fact that it is highly conserved throughout most if not all Streptococcus suis serotypes, in particular the most prevalent serotypes 1, 2, 7 and 9, that the IgM protease antigen can be used to arrive at broad cross protection among Streptococcus suis serotypes, in particular among serotypes 1, 2, 7 and 9.

WO2017/005913 (Intervacc AB) confirms the fact that the IgM protease is highly conserved throughout various Streptococcus suis serotype sand thus, the expected broad protection that can be arrived at using this antigen.

Recently patent applications regarding the use of an IgM protease antigen, in particular an IgM protease antigen of serotype 2, for protection against other serotypes, have been published. These applications confirm the cross protective nature of the IgM protease antigen.

In particular, WO 2020/094762 describes the use of an IgM protease antigen of serotype 2 against a challenge with serotype 14. It appears that very adequate protection can be obtained.

In WO 2019/115741 it is shown that the IgM protease antigen is effective to protect against a pathogenic infection with Streptococcus suis of serotype 9. However, the protection is not very high, and appears to be at best at a level obtainable with a common bacterin vaccine, i.e. a reduction of about 50% in deaths and positive blood isolation in an artificial challenge experiment (not excluding that in practice, mostly facing a less fierce challenge, protection will be at a higher level). At first glance, this somewhat disappointing protection seems to be in conflict with the high level of protection obtained with an IgM protease antigen against an infection with Streptococcus suis of serotype 9 as reported by Rieckmann et al. in Vaccine, 3 (2019) 100046 (“Vaccination with the immunoglobulin M-degrading enzyme of Streptococcus suis, IdeSsuis, leads to protection against a highly virulent serotype 9 strain”), also in an artificial challenge experiment, and the protection against Streptococcus suis of serotype 14 as shown in WO 2020/094762. Based on the art, the relatively low level of protection against a common serotype 9 bacterium cannot be understood.

Although at least some protection is expected, in the art there is no data available about the protection of an IgM protease serotype 2 against a challenge with Streptococcus suis of serotype 1 and 7.

### OBJECT OF THE INVENTION

It is an object of the invention to find an improved vaccine for providing (cross-) protection of pigs against Streptococcus suis, in particular against Streptococcus suis of various serotypes including serotype 1, 2, 7 and 9. Preferably, the vaccine comprises antigens derived from less than these 4 serotypes, yet still being capable of providing adequate protection at least against all these 4 serotypes, at least against representative strains of these serotypes as present in the field.

### SUMMARY OF THE INVENTION

In order to meet the object of the invention a vaccine has been devised comprising in combination an IgM protease antigen of Streptococcus suis serotype 7, a Streptococcus suis bacterin serotype 9, sequence type 16, and a pharmaceutically acceptable carrier.

The current invention was based on a couple of unexpected findings. Firstly, it appeared that the heterologous protection afforded by an IgM protease of serotype 2 is not as good as would be expected based on prior art teachings, in particular since the IgM protease is highly conserved among Streptococcus suis of different serotypes. In particular, as best understood, the Mac-1 domain is present to very high identity level in all Streptococcus suis serotypes known today. Still, although the homologous protection afforded by an IgM protease antigen of serotype against serotype 2 challenge is excellent, the protection against Streptococcus serotype 1 and 7 might still be markedly improved. Another unexpected finding was that the heterologous protection afforded by an IgM protease antigen of serotype 7 on its turn is very good, in particular against serotype 1 and 2. In particular the fact that the level of heterologous protection afforded against serotype 2 is markedly better than the heterologous protection afforded by serotype 2 against serotype 7 is totally unexpected.

A further highly unexpected finding was that an IgM protease antigen of serotype 2, or actually of any serotype, provides hardly any adequate protection against the most prevalent type of Streptococcus serotype 9, namely Streptococcus serotype 9, sequence type 16 (some protection is afforded, but the level is not sufficient for a commercial successful vaccine). At first glance this finding seems to be in conflict with the results as reported in Rieckmann. However, on close examination, it appears that in the Rieckmann study, a Streptococcus suis strain of serotype 9, Sequence Type 94 is used. In WO 2019/115741, although not indicated, a Streptococcus suis strain of Sequence Type 16 is used. This was found later by typing the used challenge strain according to the multilocus sequence typing as described by King et al (see above). Apparently, against the later type (S. suis of serotype 9, sequence type 16) the IgM protease antigen provides protection at a substantially lower level. The reason for this is not completely clear, but very disadvantageous, since in many countries, especially European countries such as The Netherlands, Streptococcus suis of sequence type 16 is the most prevalent (up to about 95%) pathogenic type of Streptococcus suis serotype 9 bacteria (Willemse et al. Scientific Reports, 2019, 9: 15429, “Clonal expansion of a virulent Streptococcus suis serotype 9 lineage distinguishable from carriage subpopulations”). Thus, although an IgM protease may give rise to protection across serotypes, it was found that there is a gap in effective protection in particular with respect to Streptococcus suis serotype 9, sequence type 16.

It was not until after recognizing all of the above findings, that it could be concluded that an improved vaccine that protects adequately against Streptococcus suis of serotypes 1, 2, 7 and 9 in the field (thus against the most prevalent strain types), that the current vaccine could be devised. It was found that when using an IgM protease antigen of serotype 7, adequate protection can be obtained against serotypes 1, 2 and 7, at a level better than obtainable with an IgM protease of serotype 2 as used in the art. Also, it was found that the gap in protection against serotype 9, sequence type 16 can be closed by using a Streptococcus suis bacterin of serotype 9, sequence type 16, to protect pigs against a pathogenic infection with Streptococcus suis serotype 9, sequence type 16. The IgM protease as present in the combination, although as such not suitable to provide adequate protection against Streptococcus suis of serotype 9, sequence type16, might even improve the protection of the bacterin.

With this invention, by using only two Streptococcus suis antigens of two different serotypes (7 and 9) adequate protection against the most prevalent Streptococcus suis bacteria can be obtained, closing any gaps or shortcomings in protection against Streptococcus suis bacteria when using an IgM protease of serotype 2 bacteria. The invention enables not only to arrive at the best possible protection against Streptococcus suis of serotype 9, including sequence type 16 as an important representative, but also, to arrive at a method to arrive at a very broad and high level protection across all prevalent serotypes, in particular serotypes 1, 2, 7 and 9.

The invention also pertains to a combination of an IgM protease antigen of Streptococcus suis serotype 7, and a Streptococcus suis bacterin serotype 9, sequence type 16, for use in a method to protect a pig against a pathogenic infection with Streptococcus suis.

Next to this, the invention pertains to the use of an IgM protease antigen of Streptococcus suis serotype 7 and a Streptococcus suis bacterin serotype 9, sequence type 16, for the manufacture of a vaccine for protecting pigs against a pathogenic infection with Streptococcus suis, as well as to a method for protecting pigs against a pathogenic infection with Streptococcus suis, by administering to the pigs an IgM protease antigen of Streptococcus suis serotype 7 and a Streptococcus suis bacterin serotype 9, sequence type 16.

### Definitions

An IgM protease antigen of Streptococcus suis is an enzyme that specifically degrades porcine IgM (and not porcine IgG or porcine IgA; Seele at al, in Journal of Bacteriology, 2013, 195 930-940; and in Vaccine 33:2207-2212; 5 May 2015), a protein denoted as IdeSsuis, or an immunogenic part thereof (typically having a length of at least about 30-35% of the full length enzyme). The whole enzyme has a weight of about 100-125 kDa, corresponding to about 1000-1150 amino acids, the size depending on the serotype of S. suis. In WO 2015/181356 several sequences that represent an IgM protease antigen of Streptococcus suis are given, viz. SEQ ID NO:1 (also incorporated in the present application), SEQ ID NO:2, SEQ ID NO:6, SEQ ID NO:7 and SEQ ID NO:5, the latter being an immunogenic part of the full length enzyme (denoted as the Mac-1 domain, i.e. amino acids 80-414 of SEQ ID NO:7). Other examples of immunogenic parts of the full length enzyme are given in WO2017/005913. A particular example of an IgM protease is the protease according to SEQ ID NO:1 of WO2015/1818356 or a protein having at least 90%, or even 91, 92, 93, 94, 95, 96, 97, 98, 99% up to 100% sequence identity in the overlapping regions. The amino acid sequence identity may be established with the BLAST program using the blastp algorithm with default parameters. It is expected that the IgM proteases of Streptococcus suis of various serotypes have a sequence identity higher than 75%, in particular expected to be 76, 77, 78, 79, 80, 81, 82, 83, 84, 85, 86, 87, 88, 90, 90, 91, 92, 93, 94, 95, 96, 97, 98, 99% up to 100%. An artificial protein, for example made to optimize yield in a recombinant production system of the antigen, may lead to a lower amino acid sequence identity such as 85%, 80%, 75%, 70%, 65, 60, 55 or even 50% compared with the whole enzyme, while maintaining the required immunogenic function, and is understood to be an IgM protease antigen of Streptococcus suis in the sense of the present invention.

A whole IgM protease antigen of Streptococcus suis is an antigen that comprises at least the Mac-1 domain, the region linked to structural functions, the CNV region, and optionally the cell adhesion region (see Example 1 for the identification of these regions in the genome of Streptococcus suis). This can be regarded as a whole IgM protease antigen since the signal peptide is believed to be missing in the naturally occurring (wild-type) secreted enzyme anyway, and the cell adhesion region is not believed to be essential for its function as a protease.

A vaccine is a constitution suitable for application to a subject, comprising one or more antigens in an immunologically effective amount (i.e. capable of stimulating the immune system of the target subject sufficiently to at least reduce the negative effects of a challenge of the wild-type micro-organisms), typically combined with a pharmaceutically acceptable carrier, which upon administration to the subject induces an immune response for treating an infection, i.e. aiding in preventing, ameliorating or curing the infection or any disease or disorder arising from that infection.

A repeat in a genome or corresponding amino acid sequence is a copy (exactly the same or highly similar, for example a homologue) that is repeated one or more times in the genome or corresponding amino acid sequence of an organism. It is part of the phenomenon of Copy Number Variation in which sections of the genome are repeated. Typically, the number of repeats varies between different strains of the same organism. Copy Number Variation is a type of structural variation. It is a type of duplication event that typically affects a considerable number of base pairs, e.g. somewhere between 30 and 400 base pairs, corresponding to 10-130 amino acids.

Protection against a pathogenic infection with a micro-organism is the same as arriving at protective immunity, i.e. aiding in preventing, ameliorating or curing the pathogenic infection with that micro-organism or a disorder arising from that infection, for example to prevent or reduce of the actual infection or one or more clinical signs resulting from the pathogenic infection with the pathogen.

A bacterin is a suspension of killed bacteria for use as a vaccine.

A combination of antigens is a joined use of these (individually distinct) antigens in one vaccination strategy, either by joining the distinct antigens in one vaccine formulation or by using separate antigen formulations for concurrent administration of the separate formulations.

A combination vaccine (i.e. a vaccine comprising a combination of antigens) is one (unitary) formulation that at the same time comprises different antigens. These different antigens can be mixed in a factory to provide a so-called ready-to-use combination vaccine, or mixed right before administration or during administration (e.g. using a device having two separate chamber for the separate antigens, the content of these chambers being mixed upon using the device for administration), as long as the antigens do end up in the same formulation.

A pig is any of the animals that belong to the family of Suidae.

A pharmaceutically acceptable carrier is a biocompatible medium, viz. a medium that after administration does not induce significant adverse reactions in the treated subject, capable of presenting the antigen to the immune system of the subject after administration of the composition comprising the carrier. Such a pharmaceutically acceptable carrier may for example be a liquid containing water and/or any other biocompatible solvent or a solid carrier such as commonly used to obtain freeze-dried vaccines (based on sugars and/or proteins), optionally comprising immunostimulating agents (also called an adjuvant). Optionally other substances such as stabilisers, viscosity modifiers or other components are added depending on the intended use or required properties of the corresponding vaccine.

### FURTHER EMBODIMENTS OF THE INVENTION

In a further embodiment of the vaccine according to the invention, the IgM protease antigen of Streptococcus suis serotype 7 is a whole IgM protease antigen having at least 90% sequence identity with the corresponding naturally occurring (i.e. wild type) IgM protease of the Streptococcus suis serotype 7 bacterium. Although from the art it is known that the Mac-1 domain alone of the IgM protease (about 35%) is sufficient to provide protection, the whole antigen is believed to provide a more effective immune response. In particular the sequence identity of 90% or above, such as 91, 92, 93, 94, 95, 96, 97, 98, 99 or even 100% with the naturally occurring IgM protease is preferred to arrive at adequate homologous and heterologous protection.

In still a further embodiment of the vaccine according to the invention, the IgM protease antigen of Streptococcus suis serotype 7 comprises in its amino acid sequence less than four repeats. Structural analysis of the genome of Streptococcus suis reveals that the genome of this bacterium is prone to the phenomenon of Copy Number Variation (CNV), in which sections of the genome are repeated. In particular the repeat has similarities to known protein sequences with hydrolase activity. It was found that the IgM protease of serotype 2 differs mainly from those that provide better heterologous protection (such as serotype 1 and 7) in that the serotype 2 contains 4 repeats. It is thus believed that it is advantageous to arrive at the best possible (heterologous) protection if the number of repeats is less than 4, or even less than 3, such as for example 2.

Most preferred is an IgM protease antigen of Streptococcus suis serotype 7 of sequence type 29, which contains 2 repeats.

Although the vaccine may comprise additional Streptococcus suis antigen, such as for example an IgM protease of serotype 2, it was found that it is sufficient that the vaccine comprises no other Streptococcus suis antigens other than the IgM protease antigen of Streptococcus suis serotype 7 and the Streptococcus suis bacterin serotype 9, sequence type 16, or at most an IgM protease antigen of Streptococcus suis serotype 1. More antigen means a higher cost price and a higher risk side effects due to a higher antigen load.

In a further embodiment of a combination for use in protecting against Streptococcus suis, the protection is against a pathogenic infection with Streptococcus suis of any of the serotypes 1, 2, 7 and 9.

In still a further embodiment of a combination for use according to the invention, the method comprises administering the IgM protease antigen of Streptococcus suis serotype 7 and the Streptococcus suis bacterin serotype 9, sequence type 16 to the pig at an age of at most 35 days.

In an alternative embodiment, the method comprises administering the IgM protease antigen of Streptococcus suis serotype 7 and the Streptococcus suis bacterin serotype 9, sequence type 16 to a sow in order to protect a pig (usually a piglet) through the intake of colostrum of this sow. It is known that the IgM protease (see WO2019/193078) provides adequate and long protection for piglets when they take colostrum from a vaccinated sow. Also, protection afforded by bacterins is commonly known to be transferred to piglets via colostrum.

In an embodiment the IgM protease antigen of Streptococcus suis serotype 7 and the Streptococcus suis bacterin serotype 9, sequence type 16 have been administered twice to the sow before the piglet takes the said colostrum.

The invention will now be further illustrated using the following specific examples.

### EXAMPLES

Example 1 structural analysis of the genome of Streptococcus suis.

Example 2 studies cross protection of IgM protease serotype 2 against serotype 1.

Example 3 studies cross protection of IgM protease serotype 2 against serotype 7.

Example 4 studies cross protection of IgM protease serotype 2 against serotype 9, sequence type 16.

Example 5 studies the protection afforded by IgM protease of serotypes 1 and 7 against a challenge with serotype 1.

Example 6 studies the protection afforded by IgM protease of serotypes 1 and 7 against a challenge with serotype 2.

Example 7 studies the protection afforded by IgM protease of serotypes 1 and 7 against a challenge with serotype 7.

Example 8 studies the protection afforded by a bacterin against a challenge with serotype 9, sequence type 16.

### Example 1

In this example an analysis of the genome of Streptococcus suis provided, i.e. the part that encodes for the IgM protease, in order to show how this part of the genome is structured. For this we use the genome of Streptococcus suis of a serotype 2 bacterium, as known from WO 2015/181356, published as SEQ ID NO:1 in that patent application. The sequence is enclosed again in the sequence listing of the present patent as SEQ ID NO:1. Sequence similarity search using Needleman-Wunsch alignment (see Needleman et al 1970, Laskowski et al 1997, Apweiler et al 2000; default settings) in addition to protein annotation (PDBSum and InterPro), reveals a structure for the IgM protease genome in which 5 regions can be identified:
Region 1 (Met 1—Thr 34): a signal sequence from position 1;
Region 2 (Val 35—Glu 426): the Mac-1 domain with predicted hydrolase activity;
Region 3 (Thr 427—Pro 687): a region that is linked to structural functions (e.g. involved in proper folding) and substrate binding.
Region 4 (Thr 688—Ser 919): a region that consists of 4 repeats (1*{Thr 688—Ser 744}, 2*{Thr 745—Ser 801}, 3*{Thr 802—Ser 858}, 4*{Thr 859—Ser 919}) that have similarities to known protein sequences with hydrolase activity;
Region 5 (Thr 920—Lys 1141): contains a predicted transmembrane region indicating cell wall anchor function.

The structure for Streptococcus suis bacteria of other serotypes is largely the same, but for serotype 9, sequence type 16, substantial differences are present (indicated here below):
The signal peptide is highly conserved among Streptococcus suis strains;
The Mac-1 domain is always present, highly conserved among all known strains, including the serotype 9, sequence type 16 strains;
Region 3 linked to structural functions is always present, also highly conserved but only about half the length for serotype 9, sequence type 16;
Regarding the CNV region, the repeats are very similar among different serotypes although the number varies, typically between 2 and 6. Serotype 9, sequence type 16 has 12 repeats of a completely different type which are much shorter compared to those of the other serotypes (viz. 12 AA's as opposed to about 60) and can be subdivided into three substantially different repeats;
The cell adhesion region is also highly conserved among different serotypes, but there is virtually no amino acid sequence identity with the region of the serotype 9, sequence type 16 strains.

In short, among most serotypes and sequence types the genome is structured largely the same, the most prominent difference being the number of repeats in the CNV region. The IgM protease part of the genome of serotype 9, sequence type 16 is highly similar as far as the Mac-1 domain is concerned, but differs substantially for the remaining part.

### Example 2

### Aim of the Study

From the prior art it is known that the complete IgM protease of Streptococcus suis serotype 2 (SEQ ID NO:1) provides excellent protection against homologous challenge. Also, some cross protection against serotypes 9 and 14 is known from the art. In this example the actual level of protection with this antigen against a serotype 1 challenge is assessed. For this a strain of sequence type 13 was used, which is common type of bacterium and a good representative for this serotype in the field.

### Study Design

To start with, for assessing protection against a challenge with a serotype 1 bacterium, the only challenge model available is a model wherein 3 week old piglets are challenged. This means that for assessing the protective effect induced by an IgM protease antigen, the piglets themselves cannot be vaccinated since then the time for developing an effective immune response is expected to be too short. Therefore, for assessing protection afforded by the vaccine, sows are vaccinated pre-partum, such that the antibodies induced are transferred to the piglets via the intake of colostrum. It is known from the art (U.S. Pat. No. 10,751,403) that when an IgM protease antigen provides protection in the vaccinated animal itself, it also provides excellent protection to the offspring of vaccinated sows. In other words, the protection as seen in this (indirect) challenge model is indicative for the protection provided in the vaccinated animals themselves, next to of course protection provided to piglets via the intake of colostrum of vaccinated sows.

For this study 10 pregnant sows were used, divided over 2 groups of 5 sows each. One group was vaccinated with the subunit vaccine, comprising a recombinant rIdeSsuis IgM protease antigen of serotype 2 (Seele et al: Vaccine 33:2207-2212; 5 May 2015, par. 2.2.) at 80 pg per dose in oil-in-water adjuvant (μDiluvac Forte, MSD Animal Health), at 6 weeks and 2 weeks before anticipated delivery, and one group was left as unvaccinated control group. After delivery, at 3 weeks of age, 10 piglets from vaccinated sows and 10 piglets from control sows (each group contained 2 piglets per sow) were selected for challenge. The piglets (2×10, vaccinates and controls) were challenged intra-tracheally with 10 ml challenge inoculum (aiming at 5.0×10¹⁰ CFU/ml) using a catheter or (if that was not possible) alternatively by using trans-tracheal injection. After challenge, the piglets were observed daily for clinical signs of S. suis infection such as depression, locomotory problems and/or neurological signs and scored using a regular scoring system going from 0 (no signs) to 3 for severe cases. Animals reaching the humane endpoint were euthanized. At regular times before and after vaccination (10 sows) and just before challenge (20 piglets) serum blood was collected for antibody determination. At regular times before and after challenge (20 piglets) heparin blood was collected for re-isolation of the challenge strain. At the end of the study (i.e. 11 days after challenge) all surviving piglets were euthanized.

### Results

None of the vaccines induced any unacceptable site (i.e local) or systemic reactions and thus could be considered safe. The post challenge data for the period before euthanisation are indicated in Table 1.

TABLE 1
Post challenge data

| | Average clinical | Dead after | Survival time | Positive blood |
|---|---|---|---|---|
| Group | score | challenge | (days) | isolation |
| 1 | 50.8 | 7/10 | 5.2 | 10/10 |
| 2 | 30.6 | 5/10 | 7.5 | 9/10 |

### Conclusion

The IgM protease of serotype 2 does not afford protection against a challenge with a serotype 1 Streptococcus suis bacterium.

### Example 3

### Aim of the Study

In this example the actual level of protection with the same antigen as used in Example 2 (IgM protease of serotype 2) against a serotype 7 challenge is assessed. For this a strain of sequence type 29 was used, which is common type of bacterium and representative for this serotype in the field.

### Study Design

As with serotype 1, for assessing protection against a challenge with a serotype 7 bacterium, the only challenge model available is a model wherein 3.5 week old piglets are challenged. Therefore, also in this study sows are vaccinated pre-partum, such that the antibodies induced are transferred to the piglets via the intake of colostrum.

For this study 10 pregnant sows were used, divided over 2 groups of 5 sows each. One group was vaccinated with the subunit vaccine, comprising a recombinant rIdeSsuis IgM protease antigen of serotype 2 (Seele et al: Vaccine 33:2207-2212; 5 May 2015, par. 2.2.) at 80 pg per dose in oil-in-water adjuvant (pDiluvac Forte, MSD Animal Health), at 6 weeks and 2 weeks before anticipated delivery, and one group was left as unvaccinated control group. After delivery, at 3.5 weeks of age, 10 piglets from vaccinated sows and 10 piglets from control sows (each group contained 2 piglets per sow) were selected for challenge. The piglets (2×10, vaccinates and controls) were challenged intra-tracheally with 10 ml challenge inoculum (aiming at 1.Ox 10¹ CFU/ml).

After challenge, the piglets were observed daily for clinical signs of S. suis infection such as depression, locomotory problems and/or neurological signs and scored using a regular scoring system going from 0 (no signs) to 3 for severe cases. Animals reaching the humane endpoint were euthanized. At regular times before and after vaccination (10 sows) and just before challenge (20 piglets) serum blood was collected for antibody determination. At regular times before and after challenge (20 piglets) heparin blood was collected for reisolation of challenge strain. At the end of the study (i.e. 11 days after challenge) all surviving piglets were euthanized.

### Results

None of the vaccines induced any unacceptable site or systemic reactions and thus could be considered safe. The post challenge data for the period before euthanisation are indicated in Table 2.

TABLE 2
Post challenge data

| | Average clinical | Dead after | Survival time | Positive blood |
|---|---|---|---|---|
| Group | score | challenge | (days) | isolation |
| 1 | 13.2 | 7/10 | 7.2 | 3/10 |
| 2 | 11.4 | 5/10 | 7.4 | 4/10 |

### Conclusion

The IgM protease of serotype 2 does not afford protection against a challenge with a serotype 7 Streptococcus suis bacterium.

### Example 4

### Aim of the Study

The aim of this study was to test the actual level of protection with the same antigen as used in Examples 2 and 3 (viz. IgM protease of serotype 2) against a serotype 9 challenge, in particular a challenge with a bacterium of serotype 9, sequence type 16.

### Study Design

Twenty four 3-week-old seronegative SPF piglets were used. The piglets were allotted to two groups (evenly distributed over the different litters) of 10 piglets each. Group 1 was vaccinated twice intramuscularly at 3 and 5 weeks of age as described in examples 2 and 3 and Group 2 was left as unvaccinated challenge control group. At 7 weeks of age the pigs were challenged intra-tracheally with a virulent culture of S. suis serotype 9 as described here above. After challenge the pigs were observed daily for clinical signs of S. suis infection such as depression, locomotory problems and/or neurological signs during 10 days. Animals reaching the humane endpoint after having shown specific clinical signs (i.e. locomotory or neurological) signs were euthanized without necropsy. Animals reaching the humane endpoint without having shown specific clinical signs were euthanized and necropsied including bacteriological examination to confirm the S. suis infection. At regular times before and after challenge heparin blood was collected for reisolation of the challenge strain. On day of first vaccination (5 weeks of age) the pigs were seronegative against serotype 2 derived IgM protease.

### Results

None of the vaccines induced any unacceptable site or systemic reactions and thus could be considered safe. The post challenge data for the period before euthanisation are indicated in Table 3.

TABLE 3
Post challenge data

| | | Survival time | Dead after | Positive blood |
|---|---|---|---|---|
| Group | Clinical score | (days) | challenge | isolation |
| 1 | 54 | 3.7 | 9/12 | 8/12 |
| 2 | 45 | 4.8 | 8/12 | 9/12 |

### Conclusion

The IgM protease of serotype 2 does not afford protection against a challenge with a serotype 9, sequence type 16 Streptococcus suis bacterium.

### Example 5

### Aim of the Study

In this example the protection against a serotype 1 challenge is assessed for IgM protease antigens of a serotype 1 and a serotype 7 Streptococcus suis strains. For this, antigens were made corresponding to the IgM protease of serotype 2 as used in Examples 2, 3 and 4, i.e. using an E. coli expression system as described in the art (Seele et. al, see above). The sequence used for the IgM protease antigen of serotype 7 is shown in appended SEQ ID NO:2, whereas the sequence used for the IgM protease antigen of serotype 1 is shown in appended SEQ ID NO:3. Both sequences include, next to the Mac-1 region, the CNV region and have 2 repeats in this region. The challenge strain was the same as used in Example 2.

### Study Design

The study design was the same as that of Examples 2 and 3, albeit that in each case for the challenge piglets aged 3.5 weeks were used, and groups of 10 piglets were used. Challenge for each of the serotypes corresponded to the challenge in Examples 2 and 3. Group 1 was vaccinated with the IgM protease of serotype 1, Group 2 with that of serotype 7, and Group 3 was left as challenge control.

### Results

None of the vaccines induced any unacceptable site or systemic reactions and thus could be considered safe. The post challenge data for the period before euthanisation are indicated in Table 4.

TABLE 4
Post challenge data

| | | Survival time | Dead after | Positive blood |
|---|---|---|---|---|
| Group | Clinical score | (days) | challenge | isolation |
| 1 | 17 | 8.0 | 3/10 | 2/10 |
| 2 | 27 | 6.8 | 4/10 | 4/10 |
| 3 | 52 | 3.9 | 8/10 | 7/10 |

### Conclusion

From the data it can be concluded that the IgM protease of serotype 1, as well as the IgM protease of serotype 7 protects against a virulent challenge with a serotype 1 strain. It seems that the homologous protection afforded by serotype 1 antigen is slightly better than the heterologous protection afforded by serotype 7 antigen.

### Example 6

### Aim of the Study

In this example the protection against a serotype 2 challenge is assessed for IgM protease antigens of a serotype 1 and a serotype 7 Streptococcus suis strains. For this, the same antigens were used as in Example 5. The challenge strain was a serotype 2, sequence type1 strain, representative for strains in the field.

### Study Design

The study design was largely the same as that of Example 4. Thirty 3-week-old piglets were used. The piglets were allotted to three groups (evenly distributed over the different litters) of 10 piglets each. Groups 1 and 2 were vaccinated twice intramuscularly at 3 and 5 weeks of age with the respective subunit vaccines whereas group 3 remained unvaccinated. At 7 weeks of age the pigs were challenged intra-tracheally with a virulent culture of S. suis serotype 2 strain. During 11 days after challenge the pigs were observed daily for clinical signs of S. suis infection such as depression, locomotory problems and/or neurological signs. Animals that reached the humane endpoint (HEP) were euthanized. Just before challenge, 2 days after challenge and, if applicable, on day of HEP (just before euthanasia) heparin blood was collected for re-isolation of the challenge strain.

On the day of first vaccination the piglets were seronegative or had a very low titre in specific IgM antibody ELISA. After the vaccinations groups 1 and 2 showed good antibody responses to the IgM protease, whereas the controls remained at a very low level.

### Results

None of the vaccines induced any unacceptable site or systemic reactions and thus could be considered safe. The post challenge data for the period before euthanisation are indicated in Table 5. One animal in group 1 had to be euthanised post-challenge for non-Streptococcus suis specific reasons.

TABLE 5
Post challenge data

| | | Survival time | Dead after | Positive blood |
|---|---|---|---|---|
| Group | Clinical score | (days) | challenge | isolation |
| 1 (st1) | 13.2 | 9.0 | 2/9 | 2/9 |
| 2 (st7) | 3.5 | 10.5 | 1/10 | 1/10 |
| 3 (—) | 61.7 | 1.4 | 10/10 | 10/10 |

### Conclusion

From the data it can be concluded that the IgM protease of serotype 1, as well as the IgM protease of serotype 7 protects against a virulent challenge with a serotype 2 strain.

### Example 7

### Aim of the Study

In this example the protection against a serotype 7 challenge is assessed for IgM protease antigens of a serotype 1 and a serotype 7 Streptococcus suis strains. For this, the same antigens were used as in Examples 5 and 6. The challenge strain was a serotype 7, sequence type 29 strain, representative for strains in the field.

### Study Design

The study design was the same as that of Example 5 (apart from the challenge strain). Challenge for each of the serotypes corresponded to the challenge in Examples 2 and 3. Group 1 was vaccinated with the IgM protease of serotype 1, Group 2 with that of serotype 7, and Group 3 was left as challenge control.

### Results

None of the vaccines induced any unacceptable site or systemic reactions and thus could be considered safe. The post challenge data for the period before euthanisation are indicated in Table 6.

TABLE 6
Post challenge data

| | | Survival time | |
|---|---|---|---|
| Group | Clinical score | (days) | Dead after challenge |
| 1 (st1) | 0.4 | 11 | 0/10 |
| 2 (st7) | 2.9 | 11 | 1/10 |
| 3 (—) | 12.6 | 9.2 | 3/10 |

### Conclusion

Although the challenge appeared to be less virulent as in the previous studies, from the data it can be concluded that the IgM protease of serotype 1, as well as the IgM protease of serotype 7 protects against a virulent challenge with a serotype 7 strain.

### Example 8

### Aim of the Study

The aim of this study was find a protective antigen against a serotype 9 challenge, in particular a challenge with a bacterium of serotype 9, sequence type 16, representative for strains circulating in the field. The options assessed were a bacterin alone and a bacterin in combination with an IgM protease, which is understood in the art to improve the efficacy of a bacterin (see Seele et al, Journal of Bacteriology, p. 930-940 March 2013, Volume 195 Number 5, “Identification of a Novel Host-Specific IgM Protease in Streptococcus suis”; and confirmed in WO2015/181356)

### Study Design

The study design was the same as used in Example 4, albeit that non SPF piglets were used and allotted to three groups (evenly distributed over the different litters) of 12 piglets each. Group 1 was vaccinated twice intramuscularly at 3 and 5 weeks of age with a bacterin vaccine containing inactivated Streptococcus suis bacteria of serotype 9, sequence type 16, at a level of 2×10⁹ cells. Group 2 in addition contained the IgM protease of Example 2 at 80 μg per dose. Both vaccines were formulated in the oil-in-water adjuvant as used the other examples. Group 3 was left as unvaccinated challenge control group. At 7 weeks of age the pigs were challenged intra-tracheally with a virulent culture of S. suis serotype 9 as described here above. After challenge the pigs were observed daily for clinical signs of S. suis infection such as depression, locomotory problems and/or neurological signs during 10 days. Animals reaching the humane endpoint after having shown specific clinical signs (i.e. locomotory or neurological) signs were euthanized without necropsy. Animals reaching the humane endpoint without having shown specific clinical signs were euthanized and necropsied including bacteriological examination to confirm the S. suis infection. At regular times before and after challenge heparin blood was collected for reisolation of the challenge strain. On day of first vaccination (5 weeks of age) the pigs were seronegative against serotype 2 derived IgM protease.

### Results

None of the vaccines induced any unacceptable sitel or systemic reactions and thus could be considered safe. The post challenge data for the period before euthanisation are indicated in Table 7. One animal in group 2 had to be euthanised post-challenge for non-Streptococcus suis specific reasons.

TABLE 7
Post challenge data

| | | Survival time | Dead after | Positive blood |
|---|---|---|---|---|
| Group | Clinical score | (days) | challenge | isolation |
| 1 | 14.3 | 9.3 | 2/12 | 4/12 |
| 2 | 14.3 | 9.4 | 2/11 | 2/11 |
| 3 | 51.8 | 5.0 | 8/12 | 9/12 |

### Conclusion

Protection against a virulent challenge of Streptococcus suis of serotype 9, sequence type 16 can be provided by a bacterin of that serotype, and a bacterin in combination with an IgM protease. The two types of antigens do not negatively interfere, line with what was expected based on the prior art.

Based on the above examples, the object of the invention can be met by combining an IgM protease antigen of Streptococcus suis serotype 7 or 1, with a Streptococcus suis bacterin serotype 9, sequence type 16, in a combined vaccination strategy. Also, it is believed that the two IgM protease antigens may be combined as well if needed to arrive at better protection against both serotype 1 and 7 challenge. Also, it is reasonable to believe that the level of cross-protection has to do with the number of repeats in the CNV region of the IgM protease, because this is where the difference is between the IgM protease molecules of serotypes 1 and 7 when compared with serotype 2: serotype 1 and 7 IgM protease each have two repeats whereas serotype 2 has four. The reason for the difference in cross protection is not cleat but a lower number of repeats appears to be advantageous for arrival at better level of cross-protection.

1. A vaccine comprising in combination an IgM protease antigen of Streptococcus suis serotype 7, a Streptococcus suis bacterin serotype 9, sequence type 16, and a pharmaceutically acceptable carrier.

2. A vaccine according to claim 1, characterised in that the IgM protease antigen of Streptococcus suis serotype 7 is a whole IgM protease antigen having at least 90% sequence identity with the corresponding naturally occurring IgM protease of the Streptococcus suis serotype 7 bacterium.

3. A vaccine according to claim 1, characterised in that the IgM protease antigen of Streptococcus suis serotype 7 is a whole IgM protease antigen having at least 95% sequence identity with the corresponding naturally occurring IgM protease of the Streptococcus suis serotype 7 bacterium.

4. A vaccine according to claim 1, characterised in that the IgM protease antigen of Streptococcus suis serotype 7 comprises in its amino acid sequence less than four repeats.

5. A vaccine according to an h claim 1, characterised in that the IgM protease antigen of Streptococcus suis serotype 7 comprises in its amino acid sequence less than three repeats.

6. A vaccine according to claim 1, characterised in that the IgM protease antigen of Streptococcus suis serotype 7 comprises in its amino acid sequence two repeats.

7. A vaccine according to claim 1, characterised in that the IgM protease antigen of Streptococcus suis serotype 7 is of sequence type 29.

8. A vaccine according to claim 1, characterised in that it comprises no other Streptococcus suis antigens other than the IgM protease antigen of Streptococcus suis serotype 7 and the Streptococcus suis bacterin serotype 9, sequence type 16, or at most an IgM protease antigen of Streptococcus suis serotype 1.

9-14. (canceled)

15. A method for protecting pigs against a pathogenic infection with Streptococcus suis, comprising administering to the pigs the vaccine of claim 1.

16. The method of claim 15, wherein the protection is against a pathogenic infection with Streptococcus suis of any of the serotypes 1, 2, 7 and 9.

17. The method of claim 15, wherein the vaccine is administered to the pig at an age of at most 35 days.

18. The method of claim 15, wherein the vaccine is administered to a sow in order to protect a pig through the intake of colostrum of this sow.

19. The method of claim 18, wherein the vaccine is administered twice to the sow before the pig takes the said colostrum.
